(19) United States
(12) Plant Patent Application Publication
(10) Pub. No.: US 2003/0009795 P1
(43) Pub. Date: Jan. 9, 2003

(54) Pear tree named "Cambridge"

(76) Inventors: Rodney G. Brower, Cambridge City, IN (US); William C. Brower, Greenfield, IN (US)

Correspondence Address: Kenneth A. Gandy, Woodard, Emhardt, Naughton, Moriarty & McNett, Bank Once Center/Tower, 111 Monument Circle, Ste. 3700, Indianapolis, IN, 46204, US

(21) Appl. No.: 10/241,146

(22) Filed: Sep. 11, 2002

Related U.S. Application Data
(63) Continuation of application No. 09/639,428, filed on Aug. 15, 2000, now abandoned.

(51) Int. Cl. A01H 5/00

(52) U.S. Cl. PLT/177

(57) ABSTRACT

This invention is a new and distinct cultivar of pear tree. The tree is a seedling of unknown parentage discovered in 1969 in Cambridge City, Ind., and has been named “Cambridge”. Compared to other ornamental pears, the tree has a highly acute lateral branching structure, forming an upright, pyramidal shape. The new variety forms larger diameter lateral branches early. Its characteristics provide a strong branching structure that is resistant to damage under conditions of icing.

### SUMMARY OF THE INVENTION

Cambridge is a new and distinct variety of pear tree that was discovered in a tended block of Pyrus seedlings. More particularly, the new variety was discovered in 1969 in a block of seedlings on the property of Gaar Nurseries in Cambridge City, Ind. The seedling is of unknown seed parentage. The tree was transplanted in 1973 to an isolated location on the property of Gaar Nurseries in Cambridge City, thereby assuring its identity and allowing further evaluation.

This new cultivar produces a highly upright ornamental pear tree. After observation, the selection was asexually propagated by side-budding onto Pyrus Calleryana understocks, and the grafted material has retained the described characteristics after propagation.

This new cultivar has characteristics that render it highly resistant to damage due to icing or other loads applied to its branches. Resistance to icing damage was observed on the grounds of Gaar Nurseries, where the parent tree and trees asexually reproduced therefrom were subjected to natural icing conditions during winter in the field, alongside pear trees of similar age of the “Bradford” variety. The Bradford variety trees suffered significant damage (branch breaking) under field icing conditions which resulted in no significant damage to the Cambridge variety trees.

### BRIEF DESCRIPTION OF THE FIGURES

FIG. 1 is a color photograph showing rows of Cambridge pear trees.

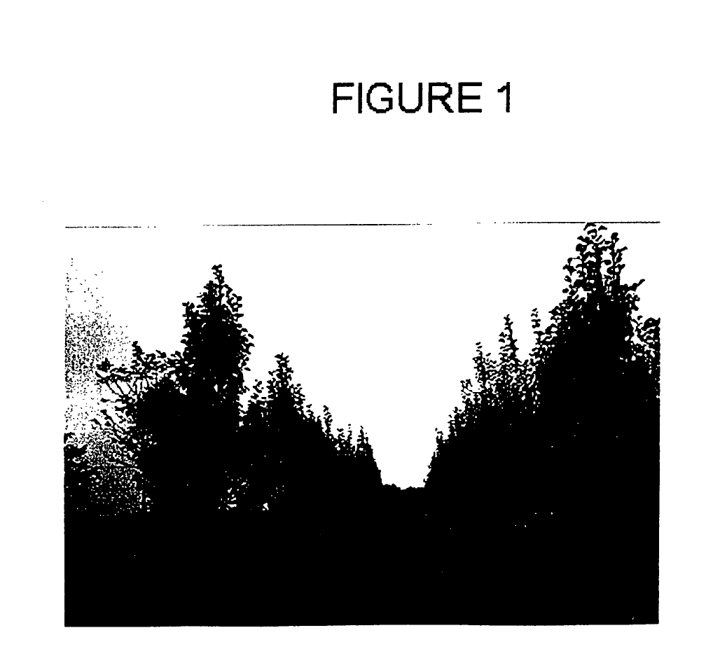

FIG. 2 is a color photograph showing the leaves of the Cambridge pear variety.

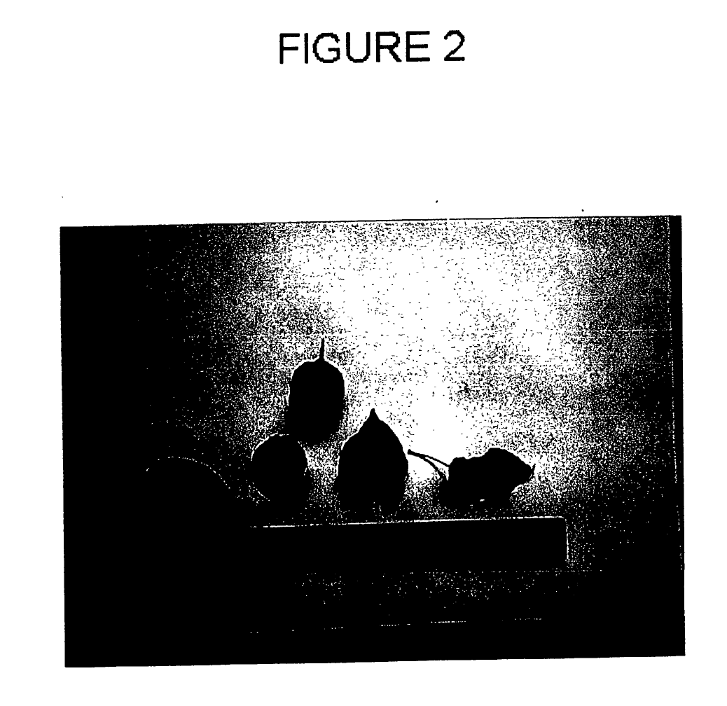

FIG. 3 is a color photograph more closely depicting the acute branching of the Cambridge pear variety.

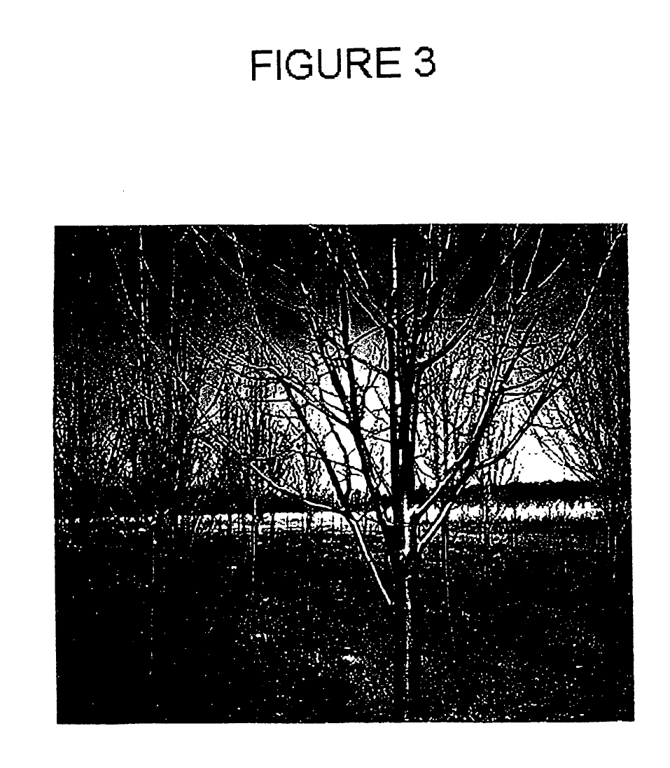

FIG. 4 is a photograph depicting rows of the Cambridge pear variety and showing fall foliage.

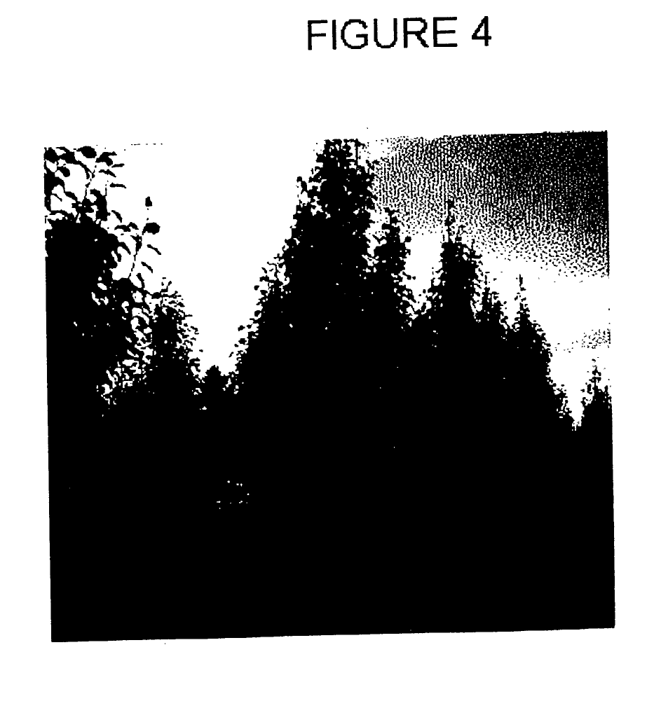

### DETAILED DESCRIPTION OF THE NEW CULTIVAR

The following is a detailed description of the new cultivar.

Tree

The Cambridge variety has good vigor, and a highly upright shape (more upright than Bradford) with a branching structure close to a dominant, straight central leader. The lateral braches extend from the central leader at a highly acute angle, significantly more acute than the Bradford variety, generally at an angle of about 18 to 28 degrees relative to the leader. The new variety grows naturally to a symmetrical form, thus having minimal pruning or training requirements to achieve an attractive, upright pyramidal shape.

Leaves

The leaves of the new variety are, in general, consistent with those found on Pyrus calleryana varieties such as the Bradford variety, with some distinct characteristics. The leaves are Broad-ovate in shape, and thick. The surfaces of the leaves are glossy, and the leaves have a generally smooth texture. The Spring and Summer foliage is green, and the Autumn foliage is bi-tone, yellow on the inside and red on the outside.

Comparison to Bradford Pear

The new variety of pear exhibits the following combination of characteristics when compared to the Bradford variety of Pyrus calleryana:

(a) exhibits a stronger branching structure, less susceptible to branch damage due to icing;

(b) exhibits better cold hardiness;

(c) forms lateral branches having a larger diameter at an earlier age; and

(d) forms lateral branches extending from the central leader at a more acute angle, providing a stronger structure and a more upright, pyramidal shape.

Propagation

The new cultivar is susceptible to asexual propagation while transmitting its characteristics from one generation to another. The new cultivar has been asexually propagated by side-budding onto Pyrus Calleryana understocks, and the grafted material retained the characteristics of the new cultivar.

I claim:

1. A new and distinct pear tree as shown and described characterized by acutely angled lateral branches, a highly upright pyramidal shape, and resistance to branch breakage under conditions of icing.
